(19) United States
(12) Patent Application Publication
(10) Pub. No.: US 2019/0366236 A1
(43) Pub. Date: Dec. 5, 2019

(54) PROCESS FOR DESALTING OF A PROTEIN SOLUTION

(71) Applicants: SANDOZ AG, Basel (CH); BOEHRINGER INGELHEIM RCV GMBH & CO KG, Wien (AT)

(72) Inventors: Nicole Walch, Wien (AT); Alois Jungbauer, Wien (AT)

(21) Appl. No.: 16/345,328

(22) PCT Filed: Nov. 10, 2017
(86) PCT No.: PCT/EP2017/078863

§ 371 Date: Apr. 26, 2019

(30) Foreign Application Priority Data
Nov. 10, 2016 (EP) 16198101.4

(51) Int. Cl. B01D 15/36; B01D 15/18; B01D 15/20; C07K 1/18

(52) U.S. Cl. CPC B01D 15/363; B01D 15/362; C07K 1/18; B01D 15/185; B01D 15/203; B01D 15/1807

(57) ABSTRACT

The present invention provides a novel process for desalting protein solutions using micropore anion and cation exchange resins. In addition, a continuous process for desalting biopharmaceutical solutions was developed. Devices for use in the process for desalting biopharmaceutical solutions were also developed.

### CROSS-REFERENCE TO RELATED APPLICATIONS

The present application is a National Phase patent application of International Patent Application Number PCT/EP2017/078863, filed on Nov. 10, 2017, which claims priority to European Patent Application No. 16198101.4, filed on Nov. 10, 2016, both of which are hereby incorporated by reference in their entireties for all purposes.

### FIELD OF THE INVENTION

The present invention generally relates to the field of biopharmaceutical bioprocessing, particularly to the reduction of salt concentrations in a biopharmaceutical solution. It covers processes of desalting a biopharmaceutical solution as well as devices for use in these processes.

### BACKGROUND OF THE INVENTION

Desalting is required in bioprocessing to interconnect unit operations which are sensitive to salt concentration and type. Many unit operations, such as protein refolding, require high salt concentrations which impede subsequent steps, such as ion exchange chromatography (IEX).

Recombinant proteins are often produced in Escherichia coli as inclusion bodies (IBs), which can easily be separated from the fermentation broth in relatively pure form and at high concentration. However, inclusion bodies consist of insoluble unfolded protein aggregates, which need to be further solubilized and refolded in order to obtain the native protein structure. The simplest and most common method of protein refolding applied in industry is resolubilization in buffers with highly chaotropic composition followed by dilution, where buffers with elevated salt concentrations are needed. Such processes result in a feed stream for the next unit operation with high salt concentrations, so that ion exchange is not a suitable option for protein recovery. Thus, either a different type of chromatography has to be used for protein capture, which might not be economical, or the salt concentration in the solution has to be reduced in order to allow efficient binding on the ion exchange resins. This reduction in conductivity is generally achieved by dilution or diafiltration.

Also, in hydrophobic interaction chromatography (HIC), proteins are bound at high salt concentrations. Elution is effected by lowering of salt, but the protein is eluting at the front of the elution gradient. Thus, protein is still present in high salt concentration and cannot be loaded on an ion-exchanger without lowering conductivity.

It is widely known that with increasing conductivity, the binding capacities of conventional ion exchangers decrease tremendously because of electrostatic shielding of the binding sites, and only moderate to low salt concentrations allow efficient capturing. In most cases this unfavorable situation is overcome either by applying affinity chromatography in the capture step which, however, is a cost-intensive resin or by introducing an additional process step such as dilution, diafiltration, or dialysis.

Another possibility are mixed ion-exchangers, which have been used for desalting of protein solutions (Hashimoto, K., S. Adachi, and Y. Shirai, Continuous Desalting of Proteins with Simulated Moving-bed Absorber. Agricultural and Biological Chemistry, 1988. 52(9): p. 2161-2167). Such mixed ion-exchangers have two major drawbacks. First, they cannot be simply regenerated and second the pH cannot be controlled. Depending on buffer composition of the protein solution, the pH will substantially drift either to acidic or basic conditions and consequently lead to a non-robust process.

Accordingly, there is a need to provide an alternative method of desalting biopharmaceutical, solutions, such as protein solutions. We have developed an integrated process, which includes deionization, washing, and regeneration in a batch or continuous mode as well as a device for use in these processes.

In addition, the invention provides a method for continuous downstream processing of protein solutions. Continuous downstream processing is still rarely applied in biopharmaceutical industry even though it carries great potential. As already practiced in other industries such as the food and chemical industries, integrated processes allow highly efficient and flexible manufacturing. Investment and operating costs can be reduced due to smaller equipment size, buffer savings, optimized cycle times and increased productivity. Even if it is currently not applied in industrial scale for the production of biopharmaceuticals, this topic has raised the interest of many researchers in academia and industry.

### SUMMARY OF INVENTION

In order to provide a desalted biopharmaceutical solution for further processing, a process was developed comprising the steps of adding the solution to a set of micropore anion and cation exchangers. In a preferred setup, the biopharmaceutical is added to a first vessel comprising a micropore anion exchanger, and then the resultant solution is transferred to a second vessel comprising a micropore cation exchanger, and the resulting desalted biopharmaceutical solution is then collected. The desalted biopharmaceutical solution resulting from the process of the invention can then either be used directly for further processing or transferred to a second set of vessels identical to the first set of vessels comprising a micropore anion exchanger and a micropore cation exchanger. This process can be repeated with one or several further sets of vessels. In addition, the vessels comprising the micropore exchange resins can be regenerated individually for reuse, the vessel with the micropore anion exchanger with NaOH and the vessel with the micropore cation exchanger with HCL. The desalted pharmaceutical can then either be applied to a new set of vessels as described above or to the regenerated vessels before collection.

This basic set-up also shown in FIG. 1a led to the development of various alternate configurations of the vessels used in the process for desalting a biopharmaceutical solution as shown in FIGS. 14 and 15. Particularly efficient was the continuous process wherein the feed of the biopharmaceutical could be redirected from the first set of vessels to a second set of vessels while the first set of vessels was regenerated, allowing for continuous flow of the biopharmaceutical feed solution and the collection of the desalted biopharmaceutical solution. This continuous setup can be expanded for use with multiple sets of vessels by adding a third, fourth, fifth, sixth or further set of vessels.

Such setups allow for a process for desalting a biopharmaceutical solution comprising the steps of (1) adding the biopharmaceutical solution (feed solution) to a first set of vessels consisting of a vessel comprising a micropore anion exchanger followed a second vessel comprising a micropore cation exchanger, (2) collecting the resulting desalted biopharmaceutical solution, (3) regenerating the micropore anion exchanger with NaOH and the cation micropore exchanger with HCL, while simultaneously adding the desalted biopharmaceutical solution from step (2) to second set of vessels identical to the first set of vessels consisting of a vessel comprising a micropore anion exchanger, followed by a vessel comprising a micropore cation exchanger. The resulting desalted biopharmaceutical solution from the second set of vessels in step (3) can be directed back to the first set of regenerated vessels and steps (1)-(3) can be repeated until the full amount of salt has been removed from the biopharmaceutical solution. Simultaneously the second set of exchangers can be regenerated, the second micropore anion exchanger with NaOH and the second cation micropore exchanger with HCL. Again, this process can be expanded to include a third, fourth, fifth, sixth or further set of vessels to be used in parallel allowing regeneration of multiple sets of vessels at the same time while loading the desalted biopharmaceutical solution on one set of regenerated vessels.

The processes described herein can use any vessel for the micropore exchanges, such as columns, or housings containing membranes or monoliths. The process can be applied to any biopharmaceutical solution that requires desalting, such as a protein solution. Preferred protein solutions are selected from the group comprising a refolding solution, a solution from hydrophobic interaction chromatography, a protein resulting from ion exchange chromatography, a solution resulting from salting out of proteins or a solution resulting from aqueous two-phase extraction. Examples of proteins in the solution are scFvs, antibodies, nobodies, bivalent antibodies, trivalent antibodies, camelid antibodies, antibody conjugates, cytokines, and peptide hormones. For further downstream processing, the desalted biopharmaceutical of the invention can be passed through to a macropore resin.

It follows that the invention also relates to a device for desalting a biopharmaceutical solution, comprising a set of vessels consisting of a vessel (1) comprising a micropore anion exchanger (AEX) connected to a vessel (2) comprising a micropore cation exchanger (CEX) by such means that the protein solution can pass from the anion exchanger into the cation exchanger and be collected after passing through the cation exchanger. In this device, the first set of vessels can be connected to a second, third, fourth or multiple identical set(s) of vessels consisting of a vessel (1) comprising a micropore anion exchanger (AEX) connected to a vessel (2) comprising a micropore cation exchanger (CEX), by such means that the protein solution can pass from the first set of vessels to the second set of vessels. Different configurations of the devices of the invention are shown in FIG. 14. Alternately, it is possible for certain biopharmaceutical solutions, to reverse the order by using a CEX followed by an AEX, as shown in FIG. 15.

### DESCRIPTION OF THE FIGURES

FIG. 1a: Scheme of continuous deionization with micropore ion exchangers through staggered cycling, called PCCC setup.

FIG. 1b: Flow scheme of continuous desalting operation with micropore ion exchanger resin in staggered cycle operation

FIG. 2: Process sequence over time. During feed the columns are interconnected, also during the initial wash step of the columns (bars in the middle) whereas regeneration and the following wash step were conducted on the single columns (separate bars).

FIG. 3: Illustration of individual steps during deionization cycle, narrow stripes represent the initial void volume of the column, which was discarded. Broad stripes represent the same volume during washing, which was collected.

FIG. 4: Chromatogram of deionization run on Marathon A2 (CV 3.7 ml) and Sepabeads (CV 6.2 ml) at 18 cm/h. Loading of scFv refolding solution (UV280=180 mAU, Cond.=11 mS/cm, pH=10.5).

FIG. 5: Chromatogram of continuous deionization run feeding 24 ml refolding solution per cycle. Grey background indicates collection of deionized refolding solution. Initial refolding sample: UV280 188 mAU, Cond. 10.0 mS/cm, pH 10.4. The red line (lowest) is conductivity, the green line (topmost) pH and the blue line (middle) UV280.

FIG. 6: Comparison of cycle time: Batch vs. Continuous mode (staggered cycling).

FIG. 7a: Screening of macropore ion exchange resins for equilibrium binding capacity of scFv A4-LCHC. Green striped: direct capturing of refolding solution with anion exchangers; Yellow solid: capturing of deionized refolding solution with cation exchangers.

FIG. 7b: List of the different macropore ion exchange resins tested

FIG. 8: Effect on pH and conductivity by blending desalted with untreated scFv refolding solution

FIG. 9: Chromatogram of analytical protein L monolith run. Impurities in the flow through peak, scFv in the elution peak

FIG. 10: Screening of micropore cation exchange resins for lowest protein binding property of scFv at refolding conditions

FIG. 11: Screening of micropore cation exchange resins for lowest protein binding property of scFv at pH 2.5

FIG. 12: Screening of micropore anion exchange resins for lowest protein binding property of scFv at refolding conditions

FIG. 13: Screening of micropore anion exchange resins for lowest protein binding property of scFv at pH 2.5

FIG. 14: Different possible desalting process configurations where anion exchange is followed by cation exchange.

FIG. 15: Different possible desalting process configurations where cation exchange is followed by anion exchange.

FIG. 16: Possible continuous desalting process configurations: A) staggered cycling using two sets of columns each consisting of anion and cation exchanger, B) PCCC with same set of columns, C) twin SMB consisting of an anion and cation SMB system with four columns each.

FIG. 17: Chromatogram of continuous desalting run feeding a hFGF-2 solution. Grey background indicates collection of deionized solution. Initial hFGF-2 solution: conductivity 44.15 mS/cm, pH 6.5.

FIG. 18: Chromatogram of continuous desalting run feeding a GFP solution. Grey background indicates collection of deionized refolding solution. Initial GFP solution: conductivity 10.0 mS/cm, pH 7.3.

FIG. 19: Chromatogram of continuous desalting run feeding redissolved mAb solution. Grey background indicates collection of deionized refolding solution. Initial mAb solution: conductivity 21.0 mS/cm, pH 7.6.

### DETAILED DESCRIPTION OF THE INVENTION

The present invention provides a novel, alternative desalting or deionizing process for biopharmaceutical solutions, preferably protein solutions, using micropore ion exchange resins as well as a device for use in the process of the invention. In the context of the present invention, the wording “deionizing” and “desalting” is used interchangeably and refers to the removal of positively and negatively charged ions present in a biopharmaceutical solution.

### Process

In the present invention, a process for performing anion (AEX) and cation exchange (CEX) was developed to remove both positively and negatively charged ions present in a biopharmaceutical solution, preferably a protein solution. In a preferred embodiment, the protein solution is a refolding solution. In the Experiments performed that led to the invention, the AEX was placed before the CEX, because it was found that in this conformation the pH transition during uptake could be controlled better and thus is more favorable for the proteins tested. However, configuration of AEX and CEX can also be swapped depending on the pH of the solution to be desalted, the amount of ions in the solution and the pH sensitivity of the target molecule. For example, plasmid DNA adsorbed and eluted from a monolithic anion exchanger medium, is present in solution with high salt concentration and possibly also at denaturing conditions. Therefore, salt concentration has to be removed. Plasmid DNA is not sensitive to low pH which is why the CEX can also be placed before the AEX in these configurations.

In a first aspect of the claimed process, two micropore columns were connected to run in series (AEX followed by CEX) so that the biopharmaceutical solution (feed stream) is added to the AEX column and the output from the AEX column is then added to the CEX column. The resulting biopharmaceutical solution, also termed desalted or deionized biopharmaceutical solution, can then be used for further processing.

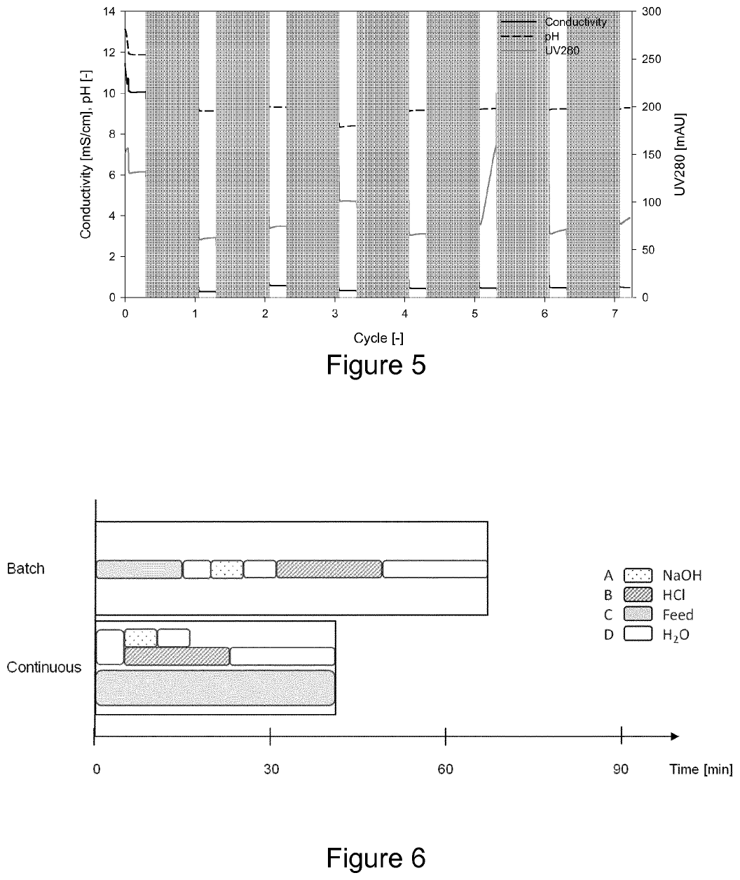

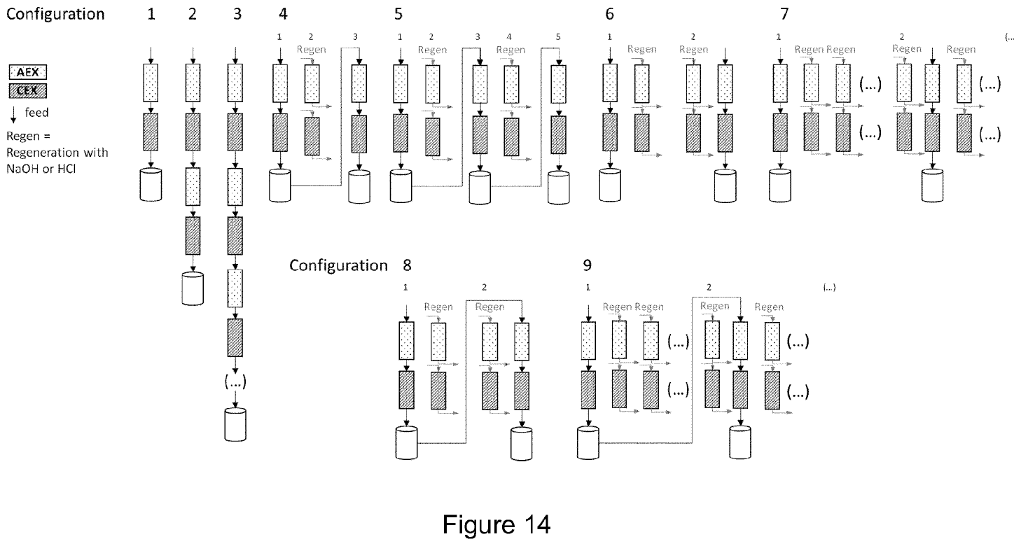

In one aspect the process underlying the invention consists of adding the high-salt biopharmaceutical solution to an AEX column, transferring the resulting solution to a CEX column and recovering the resulting desalted solution (see FIG. 14 configuration 1). However, it would be possible to use the same micropore resins in different configurations. For instance, four smaller columns in the following sequential arrangement of AEX-CEX-AEX-CEX can be used, which will prevent a sharp drop in pH value and pH excursions (see FIG. 14 configuration 2). In the current process design, the pH value of the biopharmaceutical solution initially increases due to the exchange of present anions with OH− ions, but drops then caused by the exchange of cations with H+ ions (see FIG. 4). With smaller columns connected consecutively, the pH changes will be less extreme—particularly for pH sensitive proteins this arrangement is more favorable (see FIG. 5).

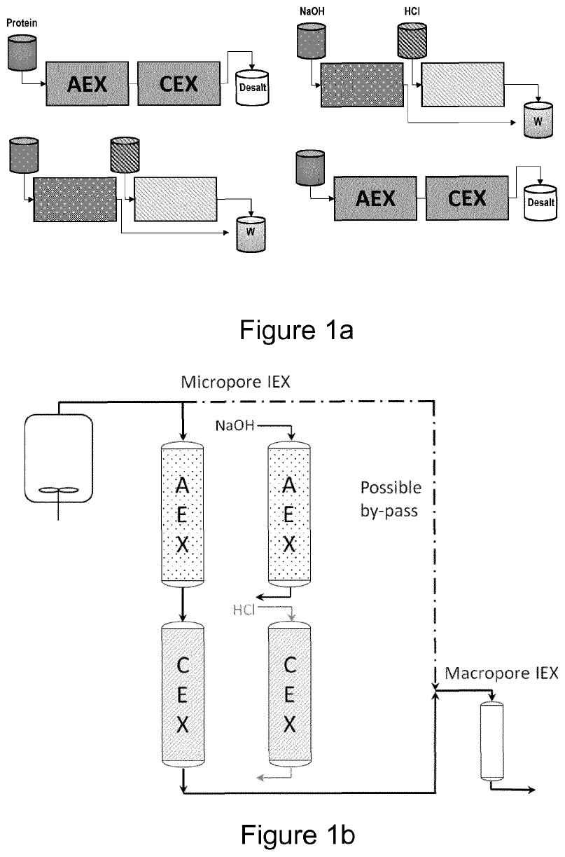

Process intensification can be achieved by operation with four, six, eight or ten or further multiples of two columns. These columns can be run continuously in staggered cycle operation in order to further reduce process time and resin volume. The intensified process set-up tested is based on two sets of columns comprising of anion and cation exchanger each. The sets are loaded alternatingly enabling continuous feeding with biopharmaceutical solution. While the biopharmaceutical solution is desalted on one set of anion and cation exchange columns, the other set(s) of columns can be washed, regenerated and re-equilibrated enabling continuous feeding of the biopharmaceutical solution. The concept of staggered cycling is illustrated schematically in FIG. 1. While loading one set of columns serially with a protein solution, the other set is regenerated individually.

This concept was realized as follows for a protein solution containing cations in excess. In order to enable continuous loading of the refolding solution, the velocity of the feed was adjusted to cover the whole duration of regeneration. The limiting step was the regeneration of the cation exchanger. Since mostly cations are present in the refolding solution a greater column volume was needed compared to the anion exchanger.

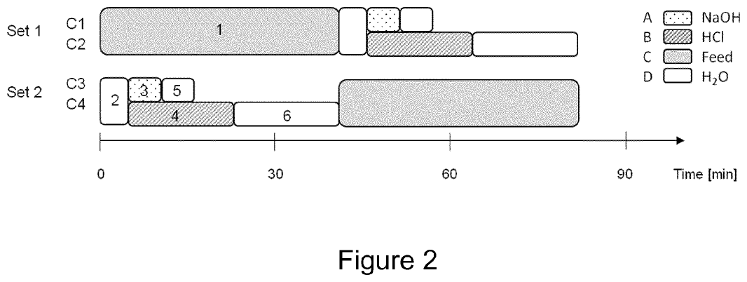

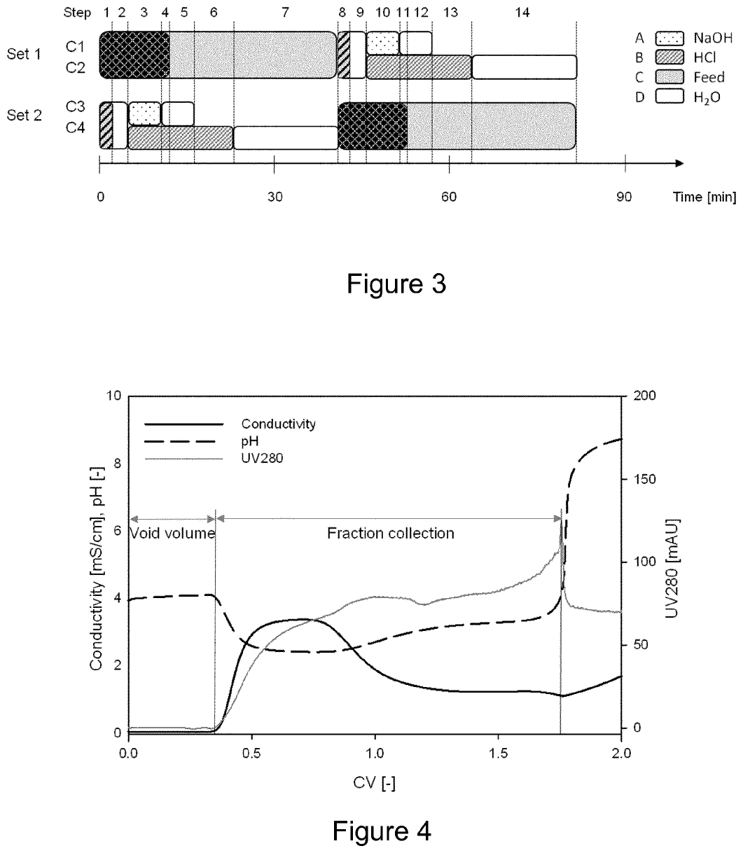

In FIGS. 2 and 3 the sequential arrangement of the process over time is shown. While loading one set of columns (1), the other set was first rinsed to wash out remaining protein from the column (2), then simultaneously the anion exchanger was regenerated with NaOH (3) and the cation exchanger with HCl (4) respectively. After regeneration, both columns were washed with 5 CV water (5/6). Afterwards, the sequence was repeated vice versa on the other set.

FIG. 4 shows a chromatogram when loading the protein solution on the two serially connected columns in batch mode. As soon as protein is recognizable by the increase in UV signal at 280 nm, collection of the desalted solution was started. A steep increase in the pH value indicated column saturation and thus the end of the deionization cycle.

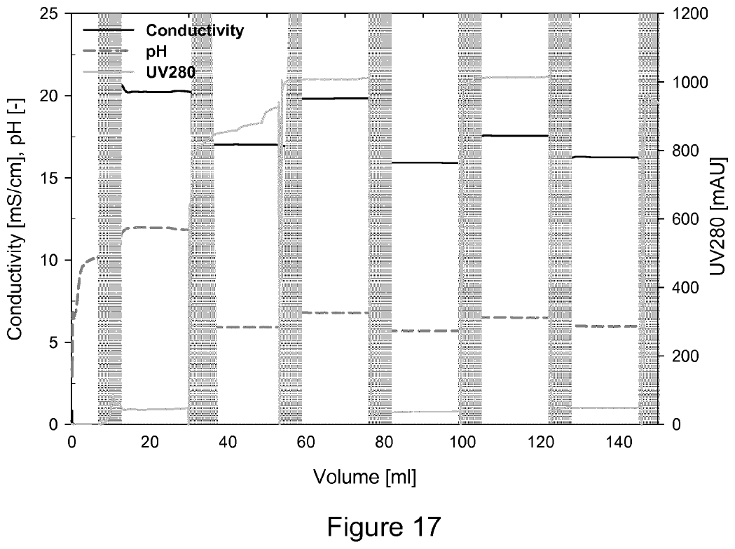

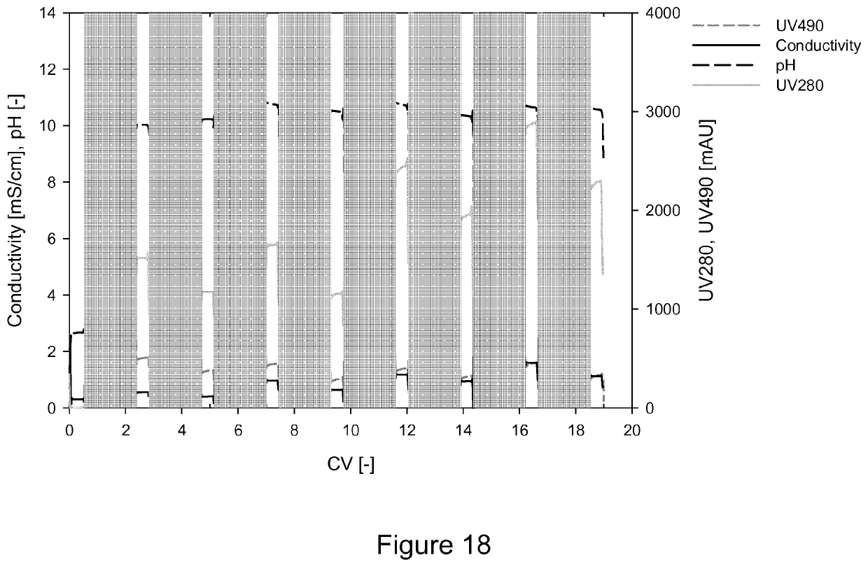

In continuous mode, it is possible to obtain deionized biopharmaceutical solution with a continuous feed stream as can be seen from FIG. 5, which is a chromatogram of continuous deionization run feeding refolding solution, as described in Example 1. A continuous desalting was conducted running the process with the micropore ion exchange columns in continuous operation. The columns were loaded uninterruptedly, but desalted solution was not collected continuously. The grey bars in FIG. 5 indicate the periods where desalted solution was collected. The conductivity throughout the whole run could be kept below 2 mS/cm and the pH value at approximately 4 during deionization with a sharp drop when the protein front leaves the column packed with microporous beads. The process employs micropore anion and cation exchangers to remove both types of ions present in the refolding solution. Regeneration of the AEX and CEX resins was achieved with HCl and NaOH, respectively. Since regeneration of the cation exchanger is the limiting process step it was necessary to reduce the flow rate of the feed to run the feed stream continuously and cover all other process steps. As in Example 5 above, in Examples 7 and 9, staggered cycle continuous desalting was also performed on GFP and on a monoclonal antibody, respectively, with the results shown in FIGS. 17 and 18, respectively. Specifically, in Example 7 the conductivity throughout the whole run could be kept below 2 mS/cm and the pH value at approximately 4 during deionization with a sharp drop when the protein front leaves the column packed with microporous beads. In Example 9, the conductivity throughout the whole run could be kept below 1 mS/cm and the pH value at approximately 4 during deionization with a sharp drop when the protein front leaves the column packed with microporous beads.

The cycle time t_{cycle} during continuous operation is defined by the longest process step. Loading the columns t_{load} has to be completed at the same time as the other process steps which is expressed in

t_{cycle}=t_{load}=t_{wash}+t_{regen}+t_{re-equilibration}

where t_{wash} is the time for washing, t_{regen} the time for regeneration and t_{re-equilibration} the time needed for rinsing the columns again with water until the pH value is stable. In our case, the limiting step was the regeneration of the cation exchanger column caused by the high concentration of cations in the refolding solution. One cycle covering loading and regeneration of one set took approximately 40 minutes in Example 5, in Examples 7 and 9 it was approximately 30 minutes. Thus, in the process of the invention, the regeneration can be achieved in 20-50 minutes, preferably between 25 and 45 minutes, most preferably in less than 35 minutes.
Comparison: Continuous Vs. Batch Mode

Running the columns continuously leads to reduced cycle time compared to batch mode. While in batch mode, the loading and regeneration steps have to be performed consecutively, in continuous operation they can be run in parallel. The whole process is restricted to the regeneration time of the cation exchanger allowing a cycle time of approximately 40 minutes according to Example 5, around 30 minutes in Examples 7 and 9. In contrast to that, in batch mode the sequence including loading, washing, regeneration and reequilibration will be completed after 66 minutes. Thus, staggered cycling improves process time by at least 38.5% and up to more than 50%. This is also demonstrated in FIG. 6.

Reduced process time implicates also improved productivity. By employing continuous operation instead of batch-wise operation, productivity can be increased from 21.8 ml/h to 35.6 ml/h. This represents a productivity increase of 163%.

In FIG. 6, the batch mode and the continuous mode are compared, and it can be seen that one run takes approximately 30-40 minutes in continuous mode while it takes over 60 minutes in batch mode. Thus, in a preferred aspect, the process of the invention is run in continuous mode. The continuous desalting process reduces cycle time compared to batch operation. While in batch mode, the loading and regeneration steps must be performed consecutively, in continuous operation they can be run in parallel. The whole process time is restricted by regeneration time of the cation exchanger allowing a cycle time of 40.5 minutes in Example 5, 32 minutes in Example 9 and 28 minutes in Example 7. In contrast to that, in batch mode when loading at 50 cm/h the sequence including loading, washing, regeneration and re-equilibration was completed after 66 minutes. Thus, staggered cycle operation improved process time by at least 38.5%. With reduced process time productivity was improved. By employing continuous operation instead of batch-wise operation, productivity was increased from 21.8 ml/h to 35.6 ml/h, which is a productivity increase to 163%. To further improve the productivity of the desalting process implementation of a third set of columns is possible. In this scenario, the second and subsequently the third set of columns are loaded while regenerating the first one. Thus, regeneration of the cation exchanger is not the limiting step any more and loading velocity can be increased to its maximum (see FIG. 14, configuration 7. Even with an additional column, the process is still simple and easy to design and scale up.

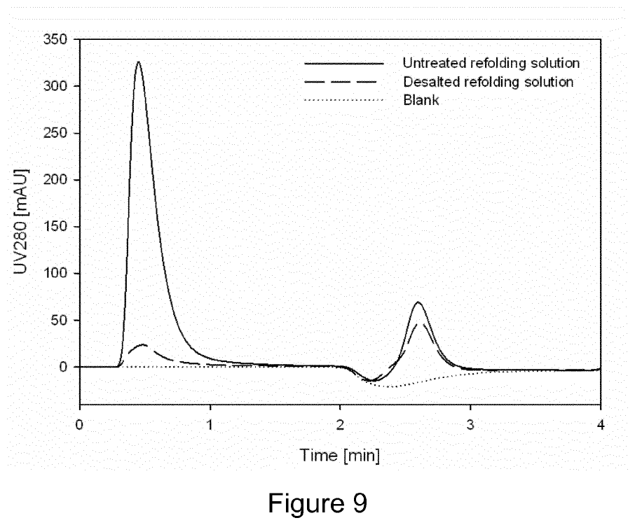

Only minimal losses of protein were observed during the desalting process according to HPLC analysis, but in addition approximately 75% of the impurities were removed compared to the initial refolding solution, so that desalting provides an additional benefit to the protein solution. In FIG. 9, the initial refolding solution is compared with the desalted protein solution. The concentration of impurities was measured by flow through and protein concentration (scFv) by the elution peak of a protein L monolith. The impurities of the initial refolding solution were considerably depleted during the desalting process. The single values can also be seen in Tables 1-3.

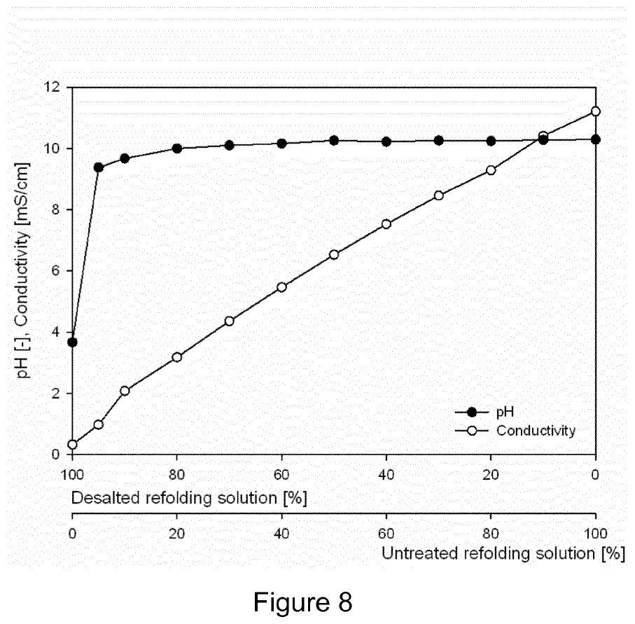

In a further aspect of the invention, it is possible to mix desalted and untreated solution before protein capture using macropore ion exchange resins. As can be seen from FIG. 8, the pH value of the protein solution jumps up to almost 10 when 10% of untreated refolding solution is blended with the desalted protein solution, conductivity rises steadily with blending of untreated refolding solution. This showcases the highly effective nature of the process of the invention. By bypassing the deionization step to a certain extent, the buffer capacity and conductivity can be controlled as required leading to a constant outlet stream.

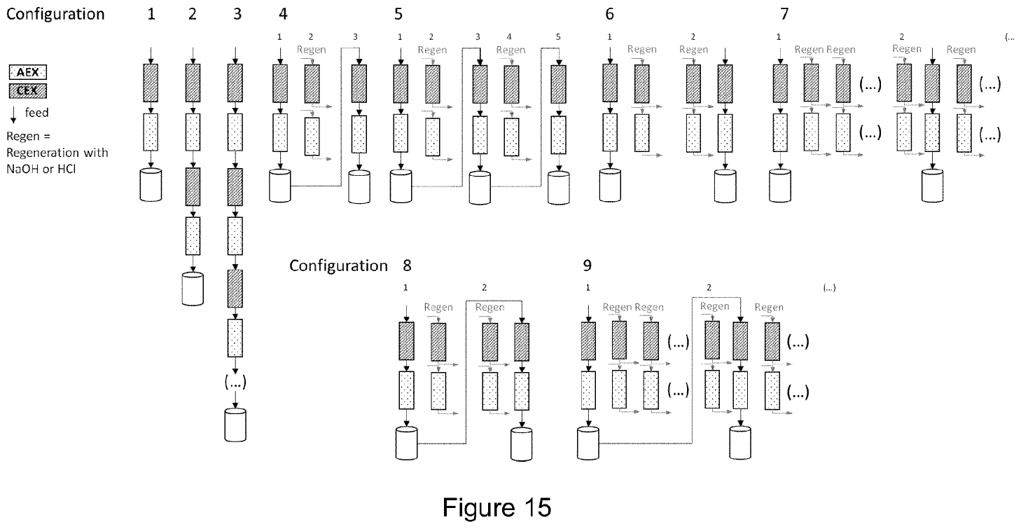

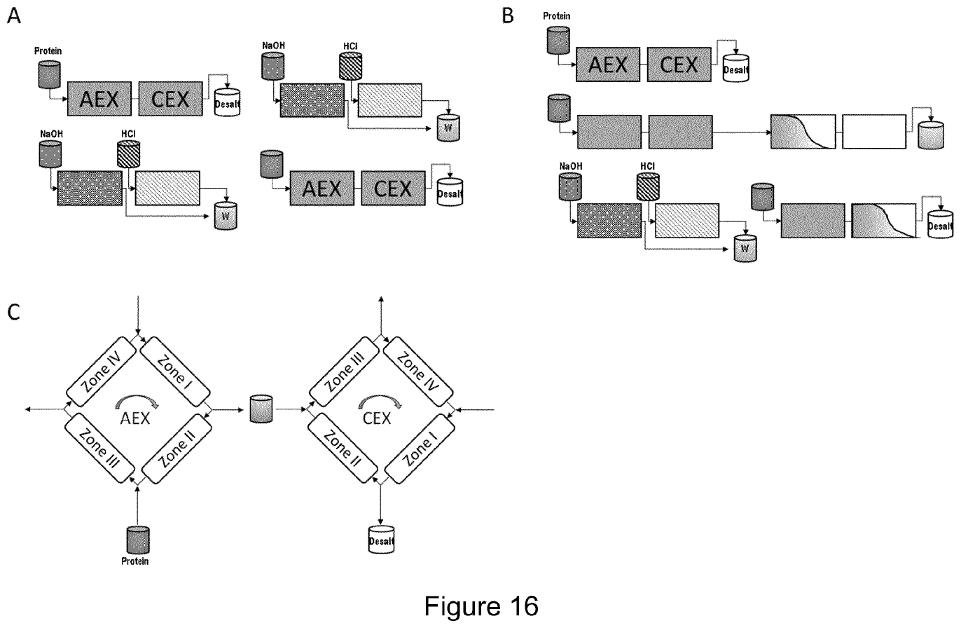

Different configurations of anion and cation exchangers are shown in FIGS. 14 and 15, and will be discussed in more detail below. These different configurations exemplify the usefulness of the process of the invention for desalting biopharmaceutical solutions.

### Biopharmaceutical Solutions

A biopharmaceutical solution for use in the process of the invention is defined as a biopharmaceutical solution having elevated salt concentration that needs to be reduced for further processing. In one embodiment the biopharmaceutical solution is a plasmid DNA solution. In a preferred embodiment the biopharmaceutical solution is a protein solution. In one aspect, the protein solution is a refolding solution (Example 1). In another aspect, the protein solution results from hydrophobic interaction chromatography (HIC), where proteins are bound at high salt concentrations. Elution is effected by lowering the salt concentration, but the protein is generally eluted at the front of the gradient and therefore still present at high salt concentrations and cannot be loaded on an IEX without lowering the conductivity (Example 5). The process of the invention can also be used to desalt protein after ion exchange chromatography (IEX, see Examples 6, 7). In a further example, in methods for crystallization and salting out proteins, solutions with high concentrations of kosmotropic salts are added. After re-dissolution of the protein the solution still has high conductivity (see Examples 8, 9). Thus, the desalting process is not only applicable for refolding solutions, but it can be employed for any protein solution where elevated salt concentrations need to be reduced for further processing. The term “elevated salt concentrations” refers to the amount of salt present in the feed solution as compared to the amount possible for the downstream bioprocess intended. For example, protein refolding solutions contain 100 mM to 1000 mM of various salts, protein are eluted from ion exchange chromatography columns at NaCl concentrations between 200 and 750 mM, and protein precipitates salted out with ammonium sulfate might be present in resolubilization solutions with 200 mM salt content. The term “untreated protein solution” refers to the feed solution that has not been subjected to desalting,

The following are examples of proteins to be desalted: a refolding solution, a solution from hydrophobic interaction chromatography, a protein resulting from ion exchange chromatography, a solution resulting from salting out of proteins, a solution resulting from aqueous two-phase extraction, and biopharmaceutical compositions comprising scFvs, antibodies, Nanobodies, bivalent antibodies, trivalent antibodies, camelid antibodies, antibody conjugates, cytokines, and peptide hormones.

### Micropore Resins

“Micropore resin” is defined as an ion exchange resin that has small pore sizes of less than 2 nm, according to IUPAC (Characterization of porous solids, E.d. S. Sing and K. Unger). This means only small molecules are able to enter the pores, while larger molecules such as proteins are excluded. Micropore ion exchange resins are commercially available and are commonly used for desalting water (Thorborg, C., H. (1971) Desalting and Purifying Water by Continuous Ion Exchange, U.S. Pat. No. 3,607,739).

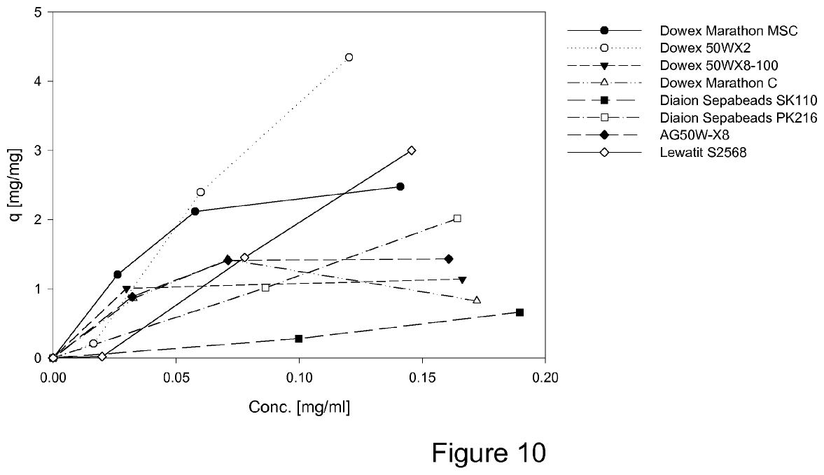

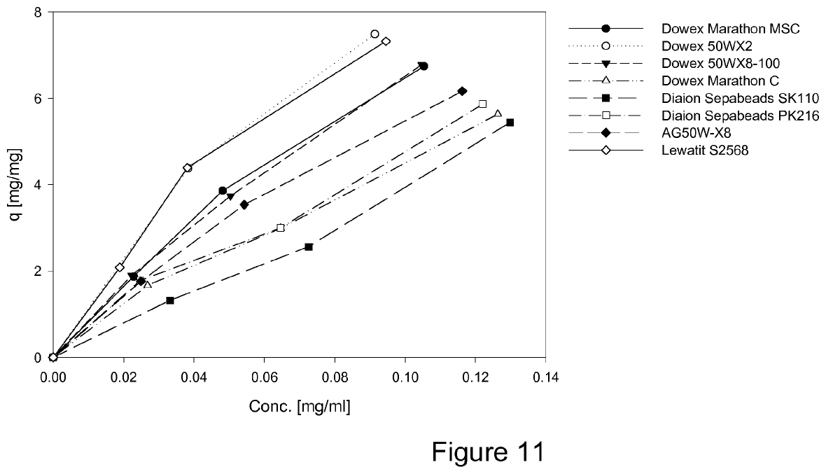

Micropore resins with low adsorption of the protein on the surface of the beads were selected by screening 19 different resins as described in Example 4. Equilibrium binding capacities were determined in order to find the resins with lowest protein binding properties. The results of these experiments can be seen in FIGS. 10-13. Specifically, since the binding behaviour changes upon pH transition, the experiments were performed at both high pH value (10.5 as in original refolding solution) and pH 2.5 (approximate pH value after deionization). FIG. 10 and FIG. 11 show the screening results of the different micropore cation exchangers.

The graphs demonstrate, that in both cases, pH 10.5 and pH 2.5 most protein was bound to Dowex 50WX2 showing least favourable behaviour. Also, the resin from Lanxess (Lewatit S2568) show rather high protein adsorption property. In contrast to that, Diaion Sepabeads SK110 from Mitsubishi adsorbed least protein in both cases and thus performed clearly best among the tested resins.

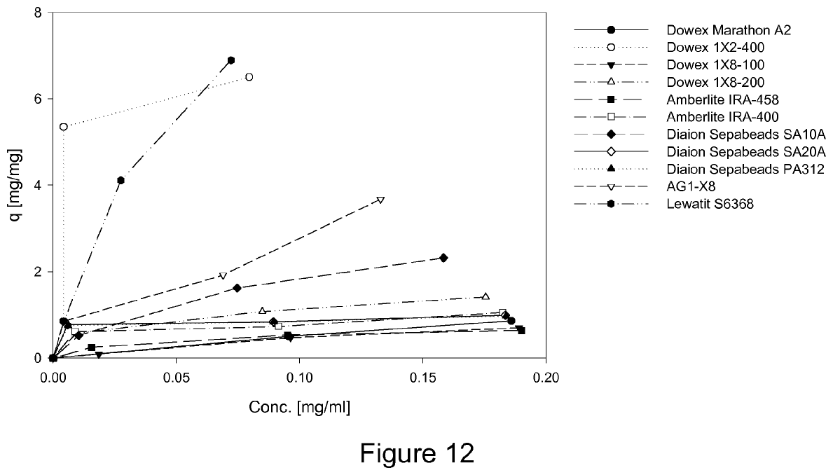

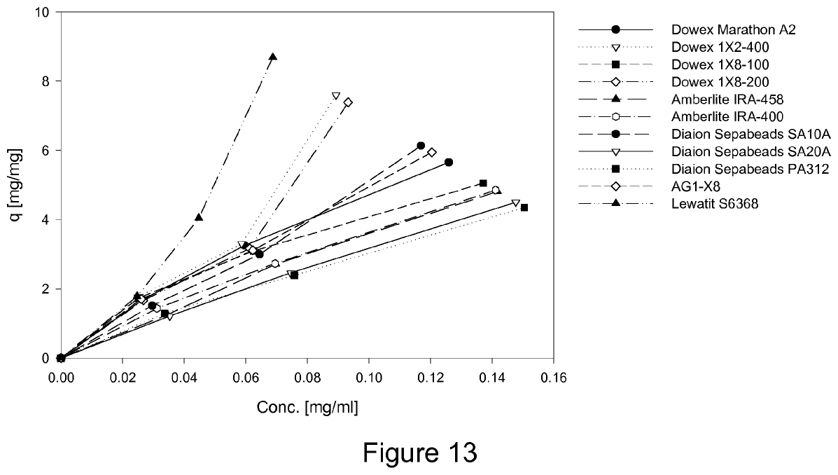

While a group of several positively charged micropore resins performed similarly well for anion removal (AEX) as can be seen from FIGS. 12 and 13, it was decided to conduct the experiments described below with Marathon A2 Micropore resins (Dow Chemical Company, Germany), a strong base anion exchange resin, was used for anion removal. This gel type material was based on styrene-divinylbenzene with dimethylethanol amine as functional groups with ion exchange capacity of 1.2 eq/l. The mean bead size is 500-600 μm with a pore size of 1.0-1.5 nm, the bulk density is 690 g/l and the quoted water retention is 45-54%. This resin was delivered in chloride form. By rinsing with a 4% (v/v) sodium hydroxide solution the hydrogen form was obtained. However, the other resins tested in Example 4 and listed in FIGS. 3 and 4, with the exception of Lewatit S6368 (Lanxess) and Dowex 1X2-400, would be equally suitable.

As the negatively charged micropore resin, Diaion Sepabeads SK110 from Mitsubishi adsorbed least protein in both cases and thus performed clearly best among the tested resins for cation removal with ion exchange capacity of >2 eq/1 (CEX). This strong acidic ion exchange material was also a gel type resin with styrene-divinylbenzene as matrix but sulfonic acid groups as active groups. The mean bead size was 400 μm with a pore size of approximately 1.0 nm, the bulk density was 845 g/l and the quoted water retention was 35-45%. This resin was delivered in sodium form. Hydrogen form was obtained by washing with 5% (v/v) hydrochloric acid.

In principle, any micropore resin as defined above can be used in the invention, assuming it does not have high protein adsorption properties.

### Macropore Resins

“Macropore resin” is defined as an ion exchange resin that has pores with a size of above 50 nm according to IUPAC (Characterization of porous solids, E.d. S. Sing and K. Unger) and allows protein diffusion into the inside of the beads. This means that the proteins are captured by the resin. For protein capture after desalting, macropore resins (IEX) can be used.

For capturing the model protein in the experiments described below, different IEX resins were tested. A comparison of equilibrium binding capacities of various macropore ion exchanger loaded with non-treated and desalted refolding solution of scFv is given in Experiment 2. Equilibrium binding capacities of various different macropore ion exchangers were determined in batch experiments. The resins were incubated with refolding samples for 24 hours at room temperature and constant movement on an end-over-end rotator (5 rpm). The amount of adsorbed protein (q) was calculated by mass balancing the initial and final protein concentration c taking into account the relationship between the volume of the mobile phase V_{mobile phase} and stationary phase Vᵣₑₛᵢₙ as per Equation

$$q = \frac{{V}_{mobilephase}}{{V}_{resin}} \cdot ( {C}_{initial} - {C}_{final} )$$

The results of this test are shown in FIG. 4.

### Effect of Deionization on Capturing

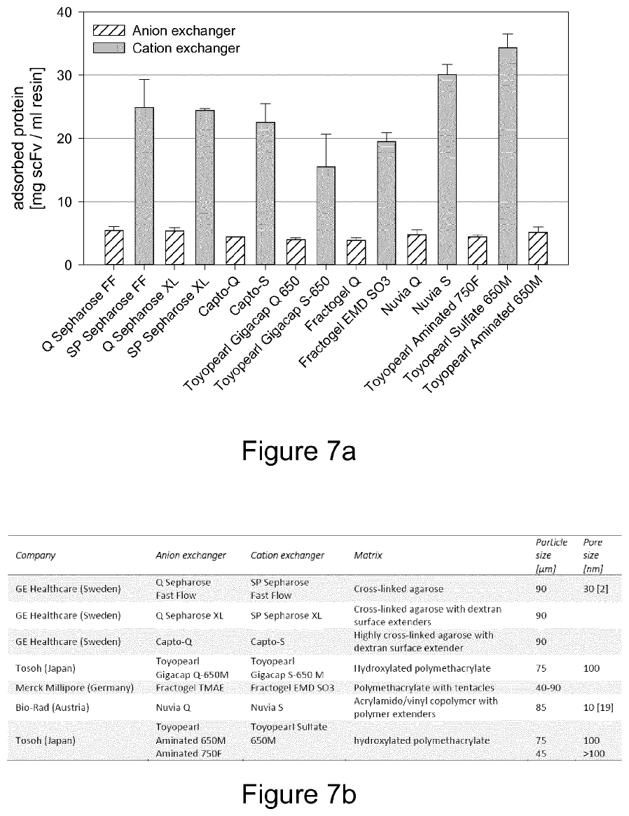

In FIG. 7a a comparison of equilibrium binding capacities of different macropore ion exchanger loaded with non-treated and deionized refolding solution of scFv is given. It illustrates, that when the refolding solution is loaded on the resin directly, q values of approximately 5 mg/ml are obtained (striped bars), whereas with reduced salt concentration, the number can be increased up to 25-35 mg/ml (solid bars). Refolding takes place at a pH value of 10.5 which is above the protein's isoelectric point. Thus, at these conditions, the protein is negatively charged and can only be bound on anion exchange resins (striped bars). Due to the pH transition during the desalting process the proteins were then present in an acidic solution below its isoelectric point. Thus, protein binding capacities of the macropore resins were tested on cation exchangers after deionization (solid bars). For macropore resins, the optimal conductivity of the sample is below 4 mS/cm.

### Devices and Alternate Configurations

The invention also relates to devices for desalting protein solutions comprising a micropore anion exchange resin connected serially to a micropore cation exchange resin. The basic setup is shown in FIG. 14C. As described in detail above, desalting according to the processes of the invention is achieved by binding of cations and anions on anion (AEX) and cation-exchange (CEX) beads. Please note that for some biopharmaceuticals, the reverse orientation, CEX followed by AEX, might be preferable. The beads or the bed can be arranged in different configurations, as described below.

In the Experiments 1-9 below, the micropore resins were packed into columns, though membranes or monoliths would be equally suitable. The size of the columns should be designed according to the concentration of anions and cations in the respective protein solution which must be removed. In Experiments 1-9 below, high resolution (HR) columns were used (1.6 cm i.d., GE Healthcare, Sweden). HR columns are for large-scale applications with high-performance media. However, any vessel that allows for packing of a micropore anion or cation exchanger would be suitable, such as a column or housing containing a membrane or monolith.

Desalting in laboratory scale is usually done in columns of a scale between 1 ml to 100 ml. But scalability of the process is possible. Pilot and industrial scale desalting can be done from 100 ml up to 2000 Liters. For example, large scale production of scFv inclusion bodies (IB) is conducted in a 1000 Liter bioreactor. 30 g/l cell dry weight and an inclusion body concentration of 3 g/l can be achieved. This results in a total IB amount of 3 kg. The IBs are suspended in 15 Liter resolubilization buffer, centrifuged and subsequently further diluted 1:20 in refolding buffer resulting in a 300 Liter refolding batch. Desalting can then be carried out on columns with a volume of 1.1 Liter anion exchanger and 3.6 Liter cation exchanger respectively.

### Stacks of AEX-CEX

The cation and anion removing function can be arranged in form of sequential beds where a cation exchange bed follows an anion exchange bed or vice versa. In this configuration regeneration is possible much more easily. The stacks can be made as a sequence of multiple columns, multiple membranes or multiple monoliths. Again, the size of the columns/membranes containing cation-exchanger or anion exchangers are determined by the amount of the cations and anions which must be removed from the protein solution.

### Possible Continuous Configurations

For continuous operation of the desalting several column arrangements are possible.

### Staggered Cycling (FIG. 1a, 15A)

While one set of columns (AEX and CEX) is loaded in serial connection, the other set is regenerated separately (as demonstrated in Examples 1, 7 and 9).

### Periodic Counter Current Chromatography (PCCC) (FIG. 15 B)

Process intensification of the above approach, where the column capacity is fully utilized by interconnecting the first set of columns to the second set until the former is completely saturated while the breakthrough is loaded on the second set.

### Simulated Moving Bed (SMB) (FIG. 15 C)

Two SMB systems would be needed for complete deionization. One system comprising of four columns of anion exchanger and the other one of cation exchanger respectively. The extract of the first system is loaded on the second one. The switch time between the different columns can be modified in a way that the buffer consumption is minimized.

### VariCol

A more flexible continuous system is represented by the VariCol technique, which is also based on a SMB principle. However, unlike a 4-zone SMB, in this process operation valves are switched asynchronously. This means there are not only one or two columns per zone at a time. Since the lines shift at different times, the column distribution between zones does not stay the same during a certain time period. By varying the zone length in time unlimited numbers of configurations are possible (see Example 8).

In sum, the processes and devices of the invention provide an efficient and inexpensive method of desalting protein solutions for further bioprocessing.

In order to investigate the potential of the application, a refolding solution of a single chain variable fragment of an antibody (scFv) expressed as IB in E. coli was used in the following Examples 1-4. First, the capacity of refolded proteins to bind to macropore ion exchangers without desalting was tested, then the protein solution was desalted by microporous ion exchangers according to the invention and then this solution was used to determine the binding capacity of the desalted protein solution on macropore ion exchangers. FIG. 7a shows that much more protein could bind to the macropore ion exchangers after desalting. In Example 5, the process of the invention was applied to protein solutions from hydrophobic interaction chromatography (HIC). In Examples 6 and 7, Green Fluorescent Protein (GFP) was desalted after ion exchange chromatography (IEX) and in Examples 8 and 9, proteins were desalted after salting out.

### EXAMPLES

### Example 1

Recombinant protein produced as inclusion body in E. coli—as described with scFv as model protein—has to be resolubilized and refolded to gain the native structure. After these two process steps high salt concentrations are present.

Refolding of Single Chain Antibody (scFv)

The scFv was over-expressed in E. coli in form of inclusion bodies (IBs). The IBs of the scFv were dissolved in resolubilization buffer containing 6 M guanidine hydrochloride (GuHCl), 100 mM Tris-Base, 5 mM EDTA, 20 mM DTT at pH 8.5. A 20% (w/v) IB suspension was made using an Ultra Turrax (Polytron PT1200C from Kinematic AG, 119 Switzerland). After incubation of 45 minutes under slight shaking, the solution was centrifuged at 12 000 g for 20 minutes at 4° C. in a bucket centrifuge (Model 5415R from Eppendorf, Germany). The supernatant was further filtered through a 0.22 μm syringe filter.

Refolding of the scFv was carried out by diluting the resolubilized IBs 20-fold in refolding buffer (3 M Urea, 0.5 M Tris, 50 mM Glycine, 2 mM Cystine, pH 10.5). To ensure maximum output the solution was incubated at 4° C. for 48 hours.

Deionization Process with Micropore Ion Exchanger

The process employs micropore anion and cation exchangers to remove both types of ions present in the refolding solution. The two columns are connected to run in series, whereupon the anion exchanger is placed first. This setup is shown in FIGS. 1a and 1b. The protein solution from the refolding tank was loaded on one set of micropore ion exchange (IEX) columns, while the other two columns were washed, regenerated and re-equilibrated individually. The desalted protein solution could then be directly captured on a macropore IEX column, or even mixed to some extent with untreated refolding solution.

Continuous operation was carried out on a Semba Octave 10 chromatography system (Semba Biosciences, USA) equipped with four Octave pumps. Four HR columns (1.6 cm diameter from GE Healthcare, Sweden) were used packed with Marathon A2, a strong micropore anion exchange resin (Dow Chemical, Germany) and Diaion Sepabeads SK110, a strong micropore cation exchange resin (Mitsubishi Chemical, Japan) respectively. Enabling online monitoring, the outlet stream E was connected to the UV detector as well as pH and conductivity probes of an Äkta Avant system (GE Healthcare, Sweden).

The column volumes (CV) were adjusted according to their dynamic binding capacities and the respective ion concentration in the refolding solution.

CV_{MarathonA2}=3.7 ml (DBC_{10%}=0.79 eq/l)

CV_{SepabeadsSK110}=12 ml (DBC_{10%}=1.26 eq/l)

Regeneration of the resins was achieved with 5% HCl and 4% NaOH respectively over 5 CV and a subsequent washing step with water over 5 CV.

Following set-up was chosen:

| | |
|---|---|
| Inlets: | A NaOH |
| | B HCl |
| | C Feed |
| | D H2O |
| Outlets: | E Deionized Refolding Solution |
| | F Waste |
| | G Waste |
| | H Waste |
| Columns | C1 Set 1 Anion Exchanger |
| | C2 Set 1 Cation Exchanger |
| | C3 Set 2 Anion Exchanger |
| | C4 Set 2 Cation Exchanger |

Regeneration and all wash steps were performed at a velocity of 100 cm/h, whereas the feed had to be adjusted. Since regeneration of the cation exchanger is the limiting process step it was necessary to reduce the flow rate of the feed to 18 cm/h to run the feed stream continuously and cover all other process steps.

The cycle time t_{cycle} during continuous operation is defined by the longest process step. Loading the columns t_{load} has to be completed at the same time as the other process steps which is expressed in

t_{cycle}=t_{load}=t_{wash}+t_{regen}+t_{re-equilibration}

where t_{wash} is the time for washing, t_{regen} the time for regeneration and t_{re-equilibration} the time needed for rinsing the columns again with water until the pH value is stable. In our case, the limiting step was the regeneration of the cation exchanger column caused by the high concentration of cations in the refolding solution. One cycle covering loading and regeneration of one set took 40.54 minutes.

The continuous deionization process covered the following steps and volumes:

| Process step | Volume | Duration |
|---|---|---|
| Feed until Vo* | 7.0 ml | 10.14 min |
| Feed until breakthrough* | 17.0 ml | 30.41 min |
| (Deionized) | | 40.54 min |
| Wash out (Deionized) | 7.0 ml | 1.79 min |
| Wash rest | 8.7 ml | 2.89 min |
| Regen NaOH | 18.5 ml | 5.52 min |
| Wash AEX | 18.5 ml | 5.52 min |
| Regen HCl | 60.0 ml | 17.91 min |
| Wash CEX | 60.0 ml | 17.91 min |
| | | 40.50 min |

*data from breakthrough experiment

In FIG. 2, the sequential arrangement of the complete process over time is shown: While loading one set of columns (1), the other set was first rinsed in serial conformation to wash out remaining protein from the column (2), then simultaneously the anion exchanger was regenerated with NaOH (3) and the cation exchanger with HCl (4). After regeneration, both columns were washed with 5 CV water (5/6). When completed, the sequence was repeated vice versa on the other set. For better understanding, the different steps were added to FIG. 2, which is illustrated in FIG. 3. FIG. 4 shows a representative chromatogram of the results of the deionization experiment.

A chromatogram of a continuous deionization run is illustrated in FIG. 5. Since the columns were not fully equilibrated at the start of the run, it took one cycle until the process steady state was achieved. As described above, the columns were loaded uninterruptedly, but deionized solution was not collected continuously. The grey bars in the graph indicate those periods where deionized solution was collected. The conductivity throughout the whole run could be kept below 2 mS/cm and the pH value at approximately 4 during deionization showing a distinctive profile. The artefacts in the UV signal are most likely due to valve switching.

Offline analyses of the pools confirmed successful deionization showing only about 0.4 mS/cm (See Table 1 below). Also, according to HPLC analysis no protein was lost during the process, but approximately 75% of the impurities were removed compared to the initial refolding sample.

TABLE 1
Analyses of pooled deionized refolding solution during continuous run

| | pH | Conductivity | scFv | Impurity |
|---|---|---|---|---|
| | [—] | [mS/cm] | [mg/ml] | removal [%] |
| Refolding | 10.4 | 10.0 | 0.10 | |
| Cycle 1 | 10.2 | 0.70 | 0.10 | 76 |
| Cycle 2 | 8.9 | 0.40 | 0.11 | 75 |
| Cycle 3 | 8.74 | 0.34 | 0.08 | 83 |
| Cycle 4 | 8.71 | 0.35 | 0.11 | 74 |
| Cycle 5 | 8.65 | 0.33 | 0.11 | 74 |
| Cycle 6 | 8.57 | 0.43 | 0.10 | 74 |
| Cycle 7 | 8.65 | 0.43 | 0.11 | 76 |

Comparison: Continuous Vs. Batch Mode

Running the columns continuously leads to reduced cycle time compared to batch mode. While in batch mode, the loading and regeneration steps have to be performed consecutively, in continuous operation they can be run in parallel. The whole process is restricted to the regeneration time of the cation exchanger allowing a cycle time of 40.5 minutes. In contrast to that, in batch mode when loading at 50 cm/h the sequence including loading, washing, regeneration and reequilibration will be completed after 66 minutes. Thus, staggered cycling improves process time by 38.5%. This is also demonstrated in FIG. 6.

Reduced process time implicates also improved productivity. By employing continuous operation instead of batch-wise operation, productivity can be increased from 21.8 ml/h to 35.6 ml/h. This represents a productivity increase to 163%.

### Example 2

### Testing of Macropore Ion Exchange Resins

Equilibrium binding capacities of various different macropore ion exchangers were determined in batch experiments. These screening experiments were carried out in Deep Well filter plates (Screening Devices, Netherlands) in combination with a vacuum manifold. Resin slurries with defined settled bed concentrations were transferred into the wells. The resins were incubated with refolding samples for 24 hours at room temperature and constant movement on an end-over-end rotator (5 rpm). The amount of adsorbed protein (q) was calculated by mass balancing the initial and final protein concentration c taking into account the relationship between the volume of the mobile phase V_{mobile phase} and stationary phase Vᵣₑₛᵢₙ as per Equation

$$q = \frac{{V}_{mobilephase}}{{V}_{resin}} \cdot ( {C}_{initial} - {C}_{final} )$$

The results of this test are shown in FIG. 4.

### Effect of Deionization on Capturing

In FIG. 7a a comparison of equilibrium binding capacities of different macropore ion exchanger loaded with non-treated and deionized refolding solution of scFv is given. It illustrates, that when the refolding solution is loaded on the resin directly, q values of approximately 5 mg/ml are obtained (striped bars), whereas with reduced salt concentration, the number can be increased up to 25-35 mg/ml (solid bars). Refolding takes place at a pH value of 10.5 which is above the protein's isoelectric point. Thus, at these conditions, the protein is negatively charged and can only be bound on anion exchange resins (striped bars). Due to the pH transition during the desalting process the proteins were then present in an acidic solution below its isoelectric point. Thus, protein binding capacities of the macropore resins were tested on cation exchangers after deionization (solid bars).

### Example 3

### Sample Analyses

Quantification of the scFv model protein from Example 1 was done by HPLC measurements on an Agilent 1100 system (Agilent, Germany) using a CIM r-protein L monolithic disk (BIA Separations, Slovenia). Bind-Elute principle was applied for the method using 30 mM Sodiumphosphate, 1M NaCl at pH 6 as running buffer and 4.5 M GuHCl (Ultrol® Grade, Merck, Germany) dissolved in running buffer as elution agent. An elution step (100% B) between minute 1 and 3 was performed enabling a total analysis time of 7 minutes at a flow rate of 1 ml/min. the injection volume was set to 100 μl and absorbance was measured at 280 nm. All samples were filtered through a 0.22 μm syringe filter prior to analysis. Offline measurements of pH value and conductivity were conducted with a Seven Multi benchtop meter (Mettler-Toledo, Switzerland).

With this method scFv concentration was determined and the relative content of impurities was assessed. The protein L ligand of the monolith binds very specifically to the kappa-light chain of antibodies, whereas other proteins do not bind and thus can be found in the flow through of the chromatogram. The method was calibrated using a scFv standard curve. The standard material was kindly provided by Boehringer Ingelheim RCV GmbH & Co KG. Purity was calculated by dividing the respective peak area by the total area.

### Example 4

### Screening of Micropore Ion Exchanger

In order to minimize product loss during the desalting step, various different micropore resins were screened. Equilibrium binding capacities were determined in order to find the resins with lowest protein binding properties. q values are based on mg adsorbed protein per mg resin dry substance.

Since the binding behaviour changes upon pH transition, the experiments were performed at both high pH value (10.5 as in original refolding solution) and pH 2.5 (approximate pH value after deionization). FIG. 10 and FIG. 11 show the screening results of the different micropore cation exchangers.

The graphs demonstrate, that in both cases, pH 10.5 and pH 2.5 most protein was bound to Dowex 50WX2 showing least favourable behaviour. Also the resin from Lanxess (Lewatit S2568) show rather high protein adsorption property. In contrast to that, Diaion Sepabeads SK110 from Mitsubishi adsorbed least protein in both cases and thus performed clearly best among the tested resins.

The same experimental set up for screening was conducted with various different micropore anion exchange resins. The findings thereof are illustrated in FIG. 12 and FIG. 13, respectively. At both pH values, the anion exchanger from Lanxess (Lewatit S6368) showed again high protein adsorption as well as Dowex 1X2-400 representing undesirable behaviour.

While a group of several resins performed similarly well at both pH values, it was decided to use Marathon A2.

### Example 5

Desalting of Protein after Hydrophobic Interaction Chromatography (HIC) Protocol 1

Soluble gamma-interferon was captured with HIC column. Elution is achieved by lowering of salt, but the protein is eluting at the front of the gradient, thus still present in high salt concentration. Recombinant human gamma-interferon (INF-γ) was expressed in E. coli BL21 in a one liter bioreactor. The cells were disrupted by a high pressure homogenizer. The homogenate was clarified by centrifugation in a bench top centrifuge. Ammonium sulfate (1 M) was added to the clarified supernatant. The formed precipitate was removed again by centrifugation and the clarified solution was loaded on a HIC column. A Toyopearl Phenyl 600 M (from Tosoh Bioscience) packed into a laboratory column (2.6 cm I.D.×100 mm). The column was equilibrated by a 1 M ammonium sulfate buffer in 25 mM Tris pH 7.5 (Buffer A). Then the conditioned supernatant was loaded on the column and after loading washed with 2 column volumes buffer A. After washing the protein was eluted with a 10 column volume linear gradient made of buffer A and buffer B (25 mM Tris pH 7.5). Elution was recorded by on-line UV-monitor at 280 nm. The eluted peak was collected and then further desalted by a mixed bed ion exchanger packed with Dowex Marathon C as cation exchanger and Diaion Sepabeads PA312 as anion exchanger in PCCC set-up (see FIG. 1). In this configuration, 2 sets of columns each consisting of an anion and a cation are employed.

### Example 6

Desalting of Protein after Hydrophobic Interaction Chromatography (HIC) Protocol 2

Soluble gamma-interferon was captured with HIC column. Elution is achieved by lowering of salt, but the protein is eluting at the front of the gradient, thus still present in high salt concentration. Recombinant human gamma-interferon (INF-γ) was expressed in E. coli BL21 in a one liter bioreactor. The cells were disrupted by a high pressure homogenizer. The homogenate was clarified by centrifugation in a bench top centrifuge. Ammonium sulfate (1 M) was added to the clarified supernatant. The formed precipitate was removed again by centrifugation and the clarified solution was loaded on a HIC column. A Toyopearl Phenyl 600 M (from Tosoh Bioscience) packed into a laboratory column (2.6 cm I.D.×100 mm). The column was equilibrated by a 1 M ammonium sulfate buffer in 25 mMTris pH 7.5 (Buffer A). Then the conditioned supernatant was loaded on the column and after loading washed with 2 column volumes buffer A. After washing the protein was eluted with a 10 column volume linear gradient made of buffer A and buffer B (25 mM Tris pH 7.5). Elution was recorded by on-line UV-monitor at 280 nm. The eluted peak was collected and then further desalted by a mixed bed ion exchanger packed with Dowex Marathon C as cation exchanger and DiaionSepabeads PA312 as anion exchanger in PCCC set-up (see FIG. 1). In this configuration, 2 sets of columns each consisting of an anion and a cation are employed.

Recombinant human fibroblast growth factor 2 (hFGF-2) was produced in E. coli BL21 (DE3) in a 10 L bioreactor. The product was expressed in soluble form in the cytosol. Cells were harvested and disrupted by high pressure homogenization. A 25% cell suspension with homogenization (50 mM Tris, 100 mM NaCl, 0.02% (v/v) Tween 20, pH 8.0) was prepared and disintegrated in a two-step homogenization applying 70 and 700 bar respectively with a GEA Panda Plus homogenizer (GEA, Germany). The homogenate was clarified by centrifugation at 18590 g for 45 minutes at 4° C. with a Heraeus Multifuge (Thermo Scientific, USA). The clarified homogenate was stored frozen at −20° C. until used.

Prior to use the clarified homogenate was slowly thawed at 4° C. overnight and again centrifuged at 18590 g (45 min at 4° C.) and filtered through a 0.22 μm filter capsule (Fluorodyne® EX EDF Membrane in Mini Kleenpak™ Capsules with 230 cm², Pall, USA).

The hFGF-2 in the homogenate was captured by a Carboxymethyl Sepharose Fast Flow (GE Healthcare, Sweden) column. A Tricorn column (GE Healthcare) with a column diameter of 10 mm and a volume of 11.8 ml was used for this purpose. The applied method is listed in Table 2. The flow rate was set to 77 cm/h.

TABLE 2
hFGF-2 capture protocol using CarboxymethylSepharose
Fast Flow with gradient elution

| METHOD BLOCK | BUFFER | DURATION |
|---|---|---|
| EQUILIBRATION | 100 mM sodium phosphate, pH 7.0 | 2 CV |
| LOAD | 0.2 μm filtered hFGF-2 homogenate | 10 CV |
| WASH | 100 mM sodium phosphate, pH 7.0 | 5 CV |
| ELUTION | Gradient from 0-1M NaCl | 2 CV |
| | (0-100% B) | |
| | 1M NaCl in Equilibration Buffer (B) | 1 CV |
| RE- | 100 mMsodium phosphate, pH 7.0 | 1 CV |
| EQUILIBRATION | | |

The elution peak was pooled and used for the next purification step based on hydrophobic interaction chromatography (HIC). A Tricorn column (GE Healthcare) packed with Toyopearl Hexyl-650C resin and a column volume of 7.9 ml (column diameter 10 mm) was used for this purpose. The applied method is listed in Table 3. The flow rate was set to 77 cm/h while loading was conducted at a flow rate of 38 cm/h.

The pH of the load material was reduced to 6.0 and 1.65 M sodium citrate was added. Prior to loading the material was also filtered through a 0.22 μm syringe filter.

TABLE 3
hFGF-2 polishing protocol using Toyopearl Hexyl-650C resin with gradient elution

| METHOD BLOCK | BUFFER | DURATION |
|---|---|---|
| EQUILIBRATION | 10 mMsodium phosphate, 1.65M sodium citrate, pH 6.0 | 2 CV |
| LOAD | 0.2 μm filtered FGF-2 capture eluate | 1 CV |
| | 1.65M sodium citrate, pH 6.0 | |
| WASH | 10 mMsodium phosphate, 1.65M sodium citrate, pH 6.0 | 3 CV |
| ELUTION | Gradient from 1.65M-0M sodium citrate (0-100% B) | 5 CV |
| | 10 mMsodium phosphate, pH 6.0 Elution Buffer (B) | 3 CV |
| RE-EQUILIBRATION | 10 mMsodium phosphate, 1.65M Sodium citrate, pH 6.0 | 1 CV |

The elution peak was pooled and used for desalting experiments. All samples were analyzed by reversed phase HPLC.

The final material had a pH of 6.5 and a conductivity of 44.15 mS/cm.

Continuous desalting operation was carried out on a Semba Octave 10 chromatography system (Semba Biosciences) equipped with four Octave pumps. Four HR columns (1.6 cm diameter from GE Healthcare) were packed with Diaion PA312 (Cl) a strong micropore anion exchange resin (Mitsubishi Chemical, Japan) and Diaion WK40L (H), a weak micropore cation exchange resin (Mitsubishi Chemical, Japan) respectively. Enabling online monitoring, the outlet stream was connected to the UV detector as well as pH and conductivity probes of an Äkta Avant system (GE Healthcare).

The column volumes were adjusted according to their binding capacities and the respective ion concentration in the hFGF-2 solution.

CV_{DiaionPA312}=8.04 ml (1.2 meq/ml)

CV_{DiaionWK40L}=4.02 ml (4.4 meq/ml)

The desalting process was operated in staggered cycle mode: while one set of columns was loaded, the second set was regenerated and vice versa.

Regeneration of the resins was achieved with 5% HCl and 4% NaOH respectively over 7 CV (NaOH) and 8 CV (HCl) and subsequent washing step with water over 8 CV (AEX) and 12 CV (CEX).

Regeneration was performed at a velocity of 100 cm/h, and the collection step at 50 cm/h, the wash steps at 200 cm/h whereas the feed was adjusted to 6.2 cm/h.

One cycle covering loading and regeneration of one set took 31.8 minutes.

A chromatogram of the continuous desalting process of the hFGF-2 solution derived from HIC polishing is shown in FIG. 17.

Offline conductivity measurement of the pools confirmed that the salt concentration in the hFGF-2 solution could be considerably lowered (See Table 4).

TABLE 4
Analyses of pooled deionized hFGF-2 solution during continuous run

| | pH | Conductivity |
|---|---|---|
| | [—] | [mS/cm] |
| hFGF-2 solution | 6.5 | 44.15 |
| Cycle 1 | 5.4 | 5.56 |
| Cycle 2 | 5.2 | 13.13 |
| Cycle 3 | 5.3 | 0.12 |
| Cycle 4 | 5.0 | 3.29 |
| Cycle 5 | 5.1 | 8.42 |
| Cycle 6 | 5.0 | 11.20 |
| Cycle 7 | 5.2 | 9.00 |
| Cycle 8 | 5.8 | 9.84 |

### Example 7

Desalting of Protein after Ion Exchange Chromatography (IEX) Protocol 1

Green fluorescent protein (GFP) is produced in E. coli and must be captured on ion exchanger. The conductivity of the clarified homogenate is too high for direct loading on an ion-exchanger in an economic manner. The elution after the capture step is performed with NaCl, protein is then present in solution with high conductivity and cannot further processed with ion-exchangers without desalting.

GFP is expressed in E. coli HMS 174 (DE3) in soluble form. The cells are disintegrated by a high pressure homogenizer. The homogenate is clarified by a centrifuge at 5000 g for 30 min. The clarified supernatant is desalted by a staggered cycling process using 2 sets of columns each consisting of 4 columns. 2 anion exchange columns packed with Amberlite IRA-458 and 2 cation exchange columns packed with Dowex Marathon MSC. The desalted protein solution is then loaded on an ion-exchange column. The column was packed with Q Sepharose FF. The column is equilibrated with a Tris buffer pH 7.5 (Buffer A). The loaded column is washed with buffer A and then eluted with a step gradient with 40% buffer B and 60% buffer A (Buffer A supplemented with 1 M NaCl). The eluted fractions are collected and then further desalted by the same set-up.

### Example 8

Desalting of Protein after Ion Exchange Chromatography (IEX) Protocol 2

Green fluorescent protein (GFP) was overexpressed in E. coli HMS 174 (DE3) in soluble form. Cells were harvested and suspended in homogenization buffer (50 mM Tris, 50 mM NaCl, pH 8.0) in a 25% slurry. A two-step homogenization was performed applying 70 and 700 bar respectively with a GEA Panda Plus homogenizer (GEA, Germany). The homogenate was clarified by centrifugation at 18590 g for 45 minutes at 4° C. with a Heraeus Multifuge (Thermo Scientific, USA). The clarified homogenate was stored frozen at −20° C. until used.

Prior to use the clarified homogenate was slowly thawed at 4° C. overnight and again centrifuged at 18590 g (45 min at 4° C.) and filtered through a 0.22 μm filter capsule (Fluorodyne® EX EDF Membrane in Mini Kleenpak™ Capsules with 230 cm², Pall, USA). The final load solution comprised of 4.1 mg/ml GFP in 50 mM Tris, 50 mM NaCl at pH 7.3 with a conductivity of 10.0 mS/cm.

Continuous operation was carried out on a Semba Octave 10 chromatography system (Semba Biosciences) equipped with four Octave pumps. Four HR columns (1.6 cm diameter from GE Healthcare) were packed with Amberlite IRA-400 (Cl) a strong micropore anion exchange resin (Sigma-Aldrich, USA) and Diaion WK40L (G), a weak micropore cation exchange resin (Mitsubishi Chemical, Japan) respectively. Enabling online monitoring, the outlet stream was connected to the UV detector as well as pH and conductivity probes of an Äkta Avant system (GE Healthcare).

The column volumes (CV) were adjusted according to their binding capacities and the respective ion concentration in the mAb solution.

CV_{Amberlite}=8.04 ml (1.4 meq/ml)

CV_{DiaionWK40L}=4.02 ml(4.4 meq/ml)

The desalting process was operated in staggered cycle mode: while one set of columns was loaded, the second set was regenerated and vice versa.

Regeneration of the resins was achieved with 5% HCl and 4% NaOH respectively over 5 CV and a subsequent washing step with water over 5 CV.

Regeneration was performed at a velocity of 100 cm/h, and the wash steps at 200 cm/h whereas the feed was adjusted to 30 cm/h.

ne cycle covering loading and regeneration of one set took 27.6 minutes. A chromatogram of the continuous desalting process of the GFP solution is shown in FIG. 18. As GFP shows a specific absorption at 490 nm, this signal is also plotted in the graph.

As in the previous experiments, the columns were loaded uninterruptedly, but deionized solution was not collected continuously. The grey bars in the graph indicate those periods where deionized solution was collected. The conductivity throughout the whole desalting run could be kept below 2 mS/cm and the pH value at approximately 3.5-4.0. Artefacts from valve switching were also noticed and can be seen as conductivity spikes in FIG. 18. These spikes are not due to a high salt concentration but are merely artifacts.

Offline analyses of the pools confirmed successful deionization (See Table 5). The GFP concentration was assessed by measuring fluorescence with an excitation wavelength of 485 nm and emission at a wavelength of 520 nm. According to these measurements, approximately 10% of the protein was lost during desalting.

TABLE 5
Analyses of pooled deionized GFP solution during continuous run

| | pH | Conductivity | GFP conc. | GFP Recovery |
|---|---|---|---|---|
| | [—] | [mS/cm] | [mg/ml] | [%] |
| GFP solution | 7.3 | 10.0 | 4.12 | |
| Cycle 1 | 9.4 | 0.49 | 3.41 | 82.8 |
| Cycle 2 | 9.6 | 0.53 | 3.59 | 87.1 |
| Cycle 3 | 9.6 | 0.98 | 3.63 | 88.1 |
| Cycle 4 | 9.6 | 1.05 | 3.69 | 89.6 |
| Cycle 5 | 9.4 | 1.54 | 3.65 | 88.6 |
| Cycle 6 | 9.7 | 1.45 | 3.95 | 95.9 |
| Cycle 7 | 9.7 | 1.72 | 3.93 | 95.4 |
| Cycle 8 | 9.7 | 1.74 | 3.89 | 94.4 |

### Example 9

Desalting of Protein after Salting Out Protocol 1

Monoclonal antibody from CHO cell culture supernatant of subclass IgG1 is salted out with 1.5 Sodium sulfate. The precipitate is harvested by microfiltration using hollow fiber microfiltration cartridges (0.45 μm pore size and 110 cm² surface area) from GE Healthcare (GE, Uppsala, Sweden). After re-dissolution of the precipitate with 25 mM Histidine puffer pH 7.0. The protein solution still has high salt concentrations and therefore further desalted by a 4 anion-exchange columns and 4 cation-exchange columns in simulated moving bed (SMB)/Varicol configuration.

### Example 10

Desalting of Protein after Salting Out Protocol 2

An anti-TNFα monoclonal antibody (mAb) was produced in a CHO K1 cell culture. Cells were removed by centrifugation (200 g for 15 minutes) and the supernatant was used further on. All centrifugation steps were carried out with a Heraeus Multifuge (Thermo Scientific, USA). Prior to use the supernatant was again centrifuged at 10,000 g for 10 minutes at 4° C.

A saturated ammonium sulfate solution was prepared with a concentration of 3.9 M at 4° C. The ammonium sulfate solution was added slowly under constant stirring at 4° C. to the mAb supernatant until a final concentration of 50% was reached within 2 hours (total working volume 4 liters). The precipitation process was allowed to take place over night at 4° C. while stirring. The precipitated pellet was separated by centrifugation at 18590 g for 30 minutes at 4° C. The supernatant was discarded and the pellet containing the antibody was redissolved in 500 ml of dissolution buffer (68.5 mM sodium chloride, 1.35 mM potassium chloride, 6 mM phosphate, pH 7.4). The resulting antibody solution had a concentration of 1.74 mg/ml, a pH value of 7.0 and a conductivity of 8 mS/cm. The antibody solution was filtered with a 0.22 μm filter (Fluorodyne® EX EDF Membrane in Mini Kleenpak™ Capsules with 230 cm², Pall, USA) before applied to the chromatographic columns.

Continuous operation was carried out on a Semba Octave 10 chromatography system (Semba Biosciences, USA) equipped with four Octave pumps. Four HR columns (1.6 cm diameter from GE Healthcare, Sweden) were packed with Amberlite IRA-400 (Cl) a strong micropore anion exchange resin (Sigma-Aldrich, USA) and Dowex Marathon MSC, a strong micropore cation exchange resin (Dow Chemical, Germany) respectively. Enabling online monitoring, the outlet stream was connected to the UV detector as well as pH and conductivity probes of an Äkta Avant system (GE Healthcare, Sweden).

The column volumes (CV) were adjusted according to their binding capacities and the respective ion concentration in the mAb solution.

CV_{Amberlite}=6.43 ml (1.4 meq/ml)

CV_{MarathonMSC}=5.63 ml (1.6 meq/ml)

The desalting process was again operated in staggered cycle mode, while one set of columns was loaded, the second set was regenerated and vice versa.

Regeneration of the resins was achieved with 5% HCl and 4% NaOH respectively over 5 CV and a subsequent washing step with water over 5 CV.

Regeneration was performed at a velocity of 100 cm/h, and the wash steps at 200 cm/h whereas the feed was adjusted to 15 cm/h.

One cycle covering loading and regeneration of one set took 31.44 minutes.

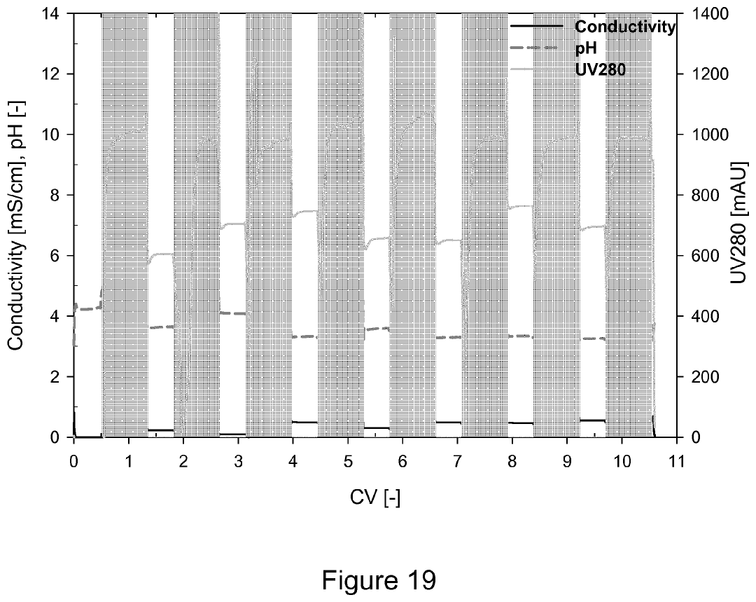

A chromatogram of the continuous desalting process of the mAb solution after salting out is shown in FIG. 19.

As in the previous experiments, the columns were loaded uninterruptedly, but deionized solution was not collected continuously. The grey bars in the graph indicate those periods where deionized solution was collected. The conductivity throughout the whole run could be kept below 1 mS/cm and the pH value at approximately 4. Artefacts from valve switching were also noticed and can be seen as conductivity spikes in FIG. 19. These spikes are not due to a high salt concentration but are merely artifacts.

Offline analyses of the pools confirmed successful deionization (See Table 6). The mAb concentration was determined by an analytical monolithic protein A CIM disk (BIA Separations, Slovenia), where also impurity content could be assessed. According to HPLC analysis only minor protein losses were recorded during the process, but approximately 60% of the impurities were depleted compared to the loaded mAb solution.

TABLE 6
Analyses of pooled deionized mAb solution during continuous run

| | | | | mAb | Impurity |
|---|---|---|---|---|---|
| | pH | Conductivity | mAb conc. | recovery | Depletion |
| | [—] | [mS/cm] | [mg/ml] | [%] | [%] |
| Redissolved | 7.00 | 21.00 | 1.74 | | |
| mAb | | | | | |
| solution | | | | | |
| Cycle 1 | 3.87 | 0.17 | 1.74 | 100.0 | 60.9 |
| Cycle 2 | 3.49 | 0.29 | 1.42 | 81.6 | 72.5 |
| Cycle 3 | 3.54 | 0.33 | 1.77 | 101.7 | 58.4 |
| Cycle 4 | 4.14 | 0.13 | 1.34 | 77.0 | 71.3 |
| Cycle 5 | 3.55 | 0.32 | 1.89 | 108.6 | 55.6 |
| Cycle 6 | 3.67 | 0.25 | 1.85 | 106.3 | 62.2 |
| Cycle 7 | 3.55 | 0.33 | 2.00 | 114.9 | 54.8 |
| Cycle 8 | 3.63 | 0.26 | 1.61 | 92.5 | 62.4 |

1. A process for desalting a protein solution comprising the steps of:
a) adding the protein solution to a first vessel (1) comprising a micropore anion exchanger,
b) transferring the solution produced in step a) to a second vessel (2) comprising a micropore cation exchanger, and
c) collecting the solution produced in step b).

2. The process according to claim 1, further comprising the steps of:
d) adding the solution from step c) to a second set of vessels identical to the first set of vessels comprising a first vessel (1) comprising a micropore anion exchanger, and a second vessel (2) comprising a micropore cation exchanger.

3. The process according to claim 1, further comprising the steps of:
e) regenerating the micropore anion exchanger in the first vessel (1) with NaOH and the cation micropore exchanger in the second vessel (2) with HCL,
f) adding the solution collected in step c) to the regenerated vessel (1) comprising the regenerated micropore anion exchanger from step e), and then transferring the resultant solution to the regenerated second vessel (2) comprising a regenerated micropore cation exchanger from step e), and
g) collecting the solution produced in step f).

4. The process according to claim 1, wherein the process is continuous and comprises the steps of:
a) adding the protein solution to a first set of vessels consisting of a vessel (1) comprising a micropore anion exchanger followed a second vessel (2) comprising a micropore cation exchanger,
b) collecting the solution produced in step a),
c) regenerating the micropore anion exchanger with NaOH and the cation micropore exchanger with HCL, while simultaneously adding the solution collected in step b) to a second set of vessels identical to the first set of vessels comprising a vessel comprising a micropore anion exchanger, followed by a vessel comprising a micropore cation exchanger,
d) collecting the solution produced in step c) from the second set of vessels,
regenerating the second micropore anion exchanger with NaOH and the second cation micropore exchanger with HCL while simultaneously directing the protein solution collected in step d) back to the first set of regenerated vessels and repeating steps a) to d) until the protein solution has been desalted.

5. The process according to claim 1 comprising the steps of:
a) adding the protein solution to a first set of vessels consisting of a vessel (1) comprising a micropore anion exchanger followed a second vessel (2) comprising a micropore cation exchanger,
b) collecting the solution produced in step a),
c) regenerating the micropore anion exchanger with NaOH and the cation micropore exchanger with HCL, while simultaneously adding the solution collected in step b) to second set of vessels identical to the first set of vessels consisting of a vessel comprising a micropore anion exchanger, followed by a vessel comprising a micropore cation exchanger,
d) collecting the solution produced in step c) from the second set of vessels,
regenerating the second micropore anion exchanger with NaOH and the second cation micropore exchanger with HCL while simultaneously directing the solution collected in step d) back to the first set of regenerated vessels and repeating steps a) to d) until the full amount of salt has been removed from the protein solution.

6. The process according to claim 1, where the protein solution is selected from the group comprising a refolding solution, a solution from hydrophobic interaction chromatography, a protein solution resulting from ion exchange chromatography, a solution resulting from salting out of proteins, and a solution resulting from aqueous two-phase extraction.

7. The process according to claim 1, further comprising the steps of adding the desalted protein solution collected from the last step to a macropore resin, and collecting the isolated protein from the macropore resin.

8. The process according to claim 7, wherein the desalted protein solution is mixed with untreated protein solution before being added to the macropore resin.

9. A device for desalting a protein solution, comprising a set of vessels comprising a vessel (1) comprising a micropore anion exchanger (AEX) connected to a vessel (2) comprising a micropore cation exchanger (CEX) in fluid communication such that the protein solution can pass from the anion exchanger into the cation exchanger and be collected after passing through the cation exchanger.

10. The device according to claim 9, wherein the first set of vessels is connected to a one or more identical sets of vessels consisting of a vessel (1) comprising a micropore anion exchanger (AEX) connected to a vessel (2) comprising a micropore cation exchanger (CEX), in fluid communication such that the protein solution can pass from the first set of vessels to the subsequent one or more sets of vessels.

11. The device according to claim 9, comprising a first set of vessels comprising a vessel (1) comprising a micropore anion exchanger (AEX) connected to a vessel (2) comprising a micropore cation exchanger (CEX) in fluid communication such that the protein solution can pass from the anion exchanger into the cation exchanger, and to one or more identical set of vessels, wherein the protein solution can be moved from the first set of vessels to the subsequent one or more identical set of vessels in series.

12. The device according to claim 9, wherein the vessels are columns packed with beads or housings containing membranes or monoliths.

13. The device according to claim 9, further comprising a vessel comprising a macropore resin connected to the last micropore cation exchanger in series.

14. A method of obtaining proteins from a protein solution comprising:
a) adding the protein solution to a vessel comprising a micropore anion resin,
b) adding the protein solution resulting from step a) to a vessel comprising a micropore cation resin,
c) adding the protein solution resulting from step b) to a vessel comprising a macropore resin;
d) recovering the protein.

15. The method according to claim 14, wherein steps a) and b) are repeated before proceeding to step c).

16. The process according to claim 2, wherein the process further comprises repeating step d) one or more times with addition set(s) of vessels.

17. The process according to claim 3, wherein the process further comprises:
h) after step g) regenerating the micropore anion exchanger in the first vessel (1) with NaOH and the cation micropore exchanger in the second vessel (2) with HCL and then i) adding the desalted protein from step g) to the regenerated micropore anion and cation exchangers from step h).

18. The process according to claim 1, wherein the protein solution comprises scFvs, antibodies, Nanobodies, bivalent antibodies, trivalent antibodies, camelid antibodies, antibody conjugates, cytokines, and peptide hormones.
